(19) United States
(12) Patent Application Publication
(10) Pub. No.: US 2006/0068484 A1
(43) Pub. Date: Mar. 30, 2006

(54) Enteropathogenic E. Coli is a separate genus pathogenic bacteria

(76) Inventor: Yakov Yevseyevich Brilman, Voorhees, NJ (US)

Correspondence Address: Yakov Brilman, Apt. 508, 219 Laurel Rd., Voorhees, NJ, 08043, US

(21) Appl. No.: 10/972,491

(22) Filed: Sep. 27, 2004

(51) Int. Cl. C12N 1/20; A61K 35/22

(52) U.S. Cl. 435/252.1; 424/545

(57) ABSTRACT

The invention is intended for treatment of diseases in accordance with immunity deficiency (HIV/AIDS diseases and other), oncological ailments, diseases connected with aggresive autoantibody (Diabetes and other autoimmune diseases), tuberculosis and etc. Purpose of the invention is the reinforcement of immunological reactivity organism. To this effect it is offered to use a method of autourinetherapy in combination with my method of autohaemovaccinoimmunotherapy and other proposed materials. Autourinetherapy differs from prototypes that use urine from sick person with accompanying adjuvant, but without it, it is subject to thermal influence under different temperatures and are entered into the organism parenteral. Under some diseases, use the urine from sick person with necessary accompaniments of raw materials.

According to existing categorizations bacteria in family Enterobacteriaceae is the genus Escherichia with type species E. Coli which are found with nonpathogenic bacteria E. Coli and pathogenic, differing from first in antigenic structure, constant and typical for each strain. Association of these bacteria in one genus, and type, species E. Coli is connected with that taken into account morphological and biochemical characteristics like them.

Tests allowing differences in these unconnected bacteria was discovered as a result of laboratory observations on significant laboratory material—this is dehydrogenase test (DGT) or oxidation-reduction test (ORT), or test of the author (Brilman's test).

In the base bacteriological researches conducted in bacteriological laboratories lies determination ferments hydrolases catalysis hydrolysis of the organic join on more simply, causing hydrolysis disinteration carbohydrate. Herewith it is not taken into account group oxidized-reconstruction ferments, being the basis of bacteriological breathing and fermentations. In this group enter aerobic and anaerobic dehydrogenases, In the last case role hydrogen acceptor in process of the oxidation execute together with other materials of different paints, which at the reaction of the join with hydrogen reduction and are decolorized. Account of the reduction of the applicable indicator lies in the basis of proposed additional test, which has such diagnostic importance either as determination other ferment, taken into account in laboratory practical (cytochrome oxidate, dehydrocarboxydase, urease, etc).

Essence of the test is concluded in the following: under incubation bacteria on semifluid ambience Giss and Kligier's medium, beside nonpathogenic Escherichia exists typical biochemical processes, under which the indicator what fixite carbohydrase fermentation the carbohydrate by change of its colouration, restores in the process of the incubations initial colour in aerobic layer of these ambiences first, but then and in anaerobic (over 48-72 h. of the incubation).

Oxidation-reduction process is a possible fix through 20-24 h. growing bacteria on nourishing ambience Giss (semifluid mannitol) on cnange the colour of the indicator in its surface layer in contrast with remaining portion of the column of the nourishing ambience. The given test is discovered in the study find beside sick children with dysfunction intestine 2886 strains bacteria E. Coli/disregarding enteropathogenic Escherichia (EPE)/and nearly beside all of them (99.38%) dehydrogenase test (DGT) or (ORT) was positive with the exclusion of 22 strains (0.62%) not refferring to known serovars/complete serotyping include somatic (o), capsular (k), flagellar (h) antigenes/enteropathogenic Escherichia (EPE) and this test was negative.

From found 112 strains of different serovars enteropathogenic Escherichia (EPE) beside all of them is the dehydrogenase test was constant negative (absence recovering the colouration of the indicator) in column semi-fluid mannitol and in Kligler's medium, that is to say also as beside Salmonellas, Shigellas, Yersinias and other pathogenic bacteria, for which typical is an absence of dehydrogenase fermentation.

Research on pathogenic intestine sticks conducts a usually applicable method and begins with serological diagnosis. However, considering that, that on Petri dish with applicable to this effect of nourishing substances enteropathogenic intestine sticks do not differ from colony conditionally pathogenic E. Coli and also that, the meet enteropathogenic intestine sticks are not diagnosed by available set of diagnostic serosities but beside which genus OR-test is negative, that are significant to their accessories to pathogenic genus of intestine sticks, follows for account of this test sift with under investigations Petri dishes on three sugar agar and semifluid mannitol (from Giss substance), from each type colonies grew on several of them, irrespective of the result of the serological research. There sift incubation under 37° C. 24-27 (before 48) hours whereupon take into account the fermenting characteristics and continue work with bacteria beside which OR-test is negative.

With provision for that constant absence dehydrogenase fermentation beside pathogenic bacteria is a typical test for them, which can enter along taken into account generic test, differentiating them from nonpathogenic bacteria allows to select enteropathogenic Escherichia Coli (EPE) in a separate pathogenic genus bacteria family Enterobacteriaceae—in genus Brilmanella.

As to discovered beside sick person with dysfunction of the bowels 22 strains bacteria (0.62%) with negative OR-test and not reffering to known serovars enteropathogenic “Escherichia Coli” (EPE) and to other pathogenic Enterobacteria, that is such bacterias need for additional study and there is possibly a variety of the pathogenic bacteria genus Brilmanella.

E. Coli having haemolytic characteristic with negative OR-test and registered by disbacteriosis also applies to a variety of bacteria of Brilmanella genus (Type “Y”).

What I claim as my invention is:

1. Method of the treatment diseases connected with immunity deficiency (HIV/AIDS diseases and etc.), aggressive autoantibody (Diabetes and other autoimmune diseases), oncological ailments, tuberculosis and etc. by using the own urine of the sick person with adding some medical preparation for reception of an autovaccine in combination with the method autohaemovaccineimmunotherapy (v. “Method of treating diseases connected with immunity deficiency”, application Ser. N.o 10/421,660) offered by me.
